(19) United States
(12) Patent Application Publication
(10) Pub. No.: US 2026/0007439 A1
(43) Pub. Date: Jan. 8, 2026

(54) Self-contouring plate system for bone fractures with fastener compression

(71) Applicant: Riviera Designs Orthopedic, Inc, Tampa, FL (US)

(72) Inventors: Islam Abdelrahman Afify, Menotia (EG); Yasser Nadim, Tampa, FL (US)

(21) Appl. No.: 19/328,307

(22) Filed: Sep. 15, 2025

Related U.S. Application Data
(63) Continuation-in-part of application No. 18/347,134, filed on Jul. 5, 2023, now Pat. No. 12,433,650.
Application No. 18/347,134 is a continuation-in-part of application No. 17/805,492, filed on Jun. 6, 2022, now Pat. No. 12,133,667.
Application No. 17/805,492 is a continuation-in-part of application No. 17/508,813, filed on Oct. 22, 2021, now abandoned.

(51) Int. Cl. A61B 17/80; A61B 17/86; A61B 17/00

(52) U.S. Cl. CPC A61B 17/8023; A61B 17/8033; A61B 17/8665; A61B 2017/00477

(57) ABSTRACT

A self-contouring plate system for bone fractures comprises a plurality of rigid plate elements connected by ball-and-socket joints. Each plate element includes a ball at one end and a socket at an opposite end for receiving a ball from an adjacent plate element. A single fastener for each plate element serves the dual function of securing the plate to bone and compressing the ball into the socket to lock the joint. The fastener includes bone threads for engaging bone and a head with a lower surface that directly contacts and compresses the ball. The system allows initial flexibility for contouring to complex bone shapes, then locks into position when the fasteners are tightened. The length is adjustable by adding or removing plate elements. Each plate element maintains substantially uniform thickness without protruding elements, and the balls are solid spherical members without through-holes to maximize strength and range of motion.

### CROSS-REFERENCE TO RELATED APPLICATIONS

This application is a continuation-in-part of U.S. Pat. App. Ser. No. 18/347,134, filed July 5, 2023, titled Self-contouring plate system for bone fractures with anti-rotation stop, which in turn is a continuation-in-part of U.S. Pat. App. Ser. No. 17/805,492, filed June 6, 2023, titled Self- contouring plate for bone fractures, which is in turn a continuation-in- part of U.S. Pat. App. Ser. No. 17/508,813, filed October 22, 2021, titled Self-contouring plate for bone fractures.

### FIELD

This invention relates to the field of treating bone fractures and more particularly to a device for treating a complex bone fracture.

### BACKGROUND

The treatment of complex bone fractures has moved beyond the antiquated treatments of full-body casts and traction.

Instead, the use of screws and plates helps surgeons to fix fractures in position, allowing the patient to regain partial mobility while the bone mends.

But the use of mechanical fracture supports, such as plates, is complicated by bones with complex shapes, such as the pelvis.

Current methods require the surgeon to contour, or bend, a plate during surgery, the plate intended to match the contour of the patient's bone.

This contouring is difficult and imperfect and can result in fractures that are only partially reduced. And imperfect contouring can cause loss of reduction obtained prior to applying the plate. The result is increased healing time and decreased patient mobility.

What is needed is a device that is contoured to the bone, the device then locked into shape and affixed to the bone.

### SUMMARY

The self-contouring plate system for bone fractures with fastener compression allows a surgeon to bridge a bone fracture, primarily in bones of complex shape where the use of plates or screws is difficult.

The self-contouring plate system is formed from a series of similar or identical rigid elements, the elements able to bend and rotate with respect to each other. This flexibility is initially helpful as the surgeon contours the device to the shape of the bone. When the desired shape is reached, the elements are locked into place.

The length of device is adjusted by adding or removing elements, much like a necklace. Each element of the self-contouring plate includes a ball that extends away from a body or plate, a cavity for receiving the ball of the neighboring plate, and one or more screws to compress the ball within the cavity.

The body of each rigid element is preferably formed from a single piece of material. The result is increased life due to a lack of joints.

Each element can rotate in three directions - swivel left and right, or yaw; tilt forward and backward, or pitch; and rotate about its centerline, or roll.

This freedom of rotation is created by a ball-and-socket connection that joins each element to the next. When the desired arrangement and angles are reached, the ball-and-socket joint is fixed in position by compression of the lower surface of a not against the head of the ball, the ball pressed into a half or partial socket.

The fastener used to compress the ball into the socket is the same fastener that is used to affix the body, or plate, to the bone of the patient. By directly contacting the head or ball with the nut or fastener used to affix each rigid element to the patient's bone, there is no need for material deformation or other types of compression. The result is a solid, rotation resistant, connection between rigid elements.

Restated, the ball-and-socket joint has both a locked position or condition, and an unlocked position or condition - a compressed position and an uncompressed position. In the preferred embodiment, compression of the socket is created by compression of a nut against an upper half of the ball, pressing the lower half of the ball into a socket, the socket being part of the plate or body of the device.

The ball-and-socket connection allows for a full range of motion. The preferred embodiment has the ability to swivel in 45 degrees of yaw, tilt between 45- and 9o-degrees of pitch, and rotate in 360 degrees of roll.

The body includes a screw hole through which the bone screw or fastener passes to fix the device to the underlying bone. The hole is threaded, interfacing with corresponding threads on the fastener as the fastener passes through the plate.

The entire device is intended for permanent internal implantation, directly against the bone. The device does not protrude through muscle or skin, and does not have elements that remain external to the body. The plates are optionally curved to match a surface profile of a curved bone.

The centerline of each element of the device is preferably consistent, with the centerline of the ball matching that of the centerline of the body. When installation is complete, there are no protruding elements that could cause discomfort by aggravating the surrounding tissues. Stated differently, in the preferred embodiment the thickness of the device is substantially consistent, without protruding elements. The ball and socket are preferably positioned at opposite ends of the body.

The self-contouring plate is strengthened by being positioned against the surface of the bone. The plates and connections are directly against the surface of the bone, avoiding rotational moments that would increase the force against the plates. This is in contrast to the prior art devices, which were placed partially outside the patient's skin, resulting in traumatic and uncomfortable pins that passed through the patient's bone and muscle.

The prior art placed the points of rotation away from the bone, thus requiring a thicker mechanism to compensate for the resulting rotational forces.

The self-contouring plate includes a solid ball, without a through-hole for a fixation screw. The result is a stronger ball connection with more material, and a stronger ball that better resists deformation from compression. The ball is preferably spherical, with the only interruption to its surface being the neck that connects the ball to the body of the plate.

Additionally, by using a solid ball, the greatest range of movement is possible. Requiring placement of a fastener through the ball limits angular rotation of the ball because the hole in the ball must line up with a second hole for receipt of the fastener.

This additional range of motion is helpful in complex fractures, such as fractures of the pelvis and acetabulum.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a first isometric view of the self-contouring plate with fastener compression.

FIG. 2 illustrates a second isometric view of the self-contouring plate with fastener compression.

FIG. 3 illustrates a third isometric view of the self-contouring plate with fastener compression.

FIG. 4 illustrates a fourth isometric view of the self-contouring plate with fastener compression.

FIG. 5 illustrates a first detail view of the self-contouring plate with fastener compression.

FIG. 6 illustrates a second detail view of the self-contouring plate with fastener compression.

FIG. 7 illustrates a third detail view of the self-contouring plate with fastener compression.

FIG. 8 illustrates a fourth detail view of the self-contouring plate with fastener compression.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

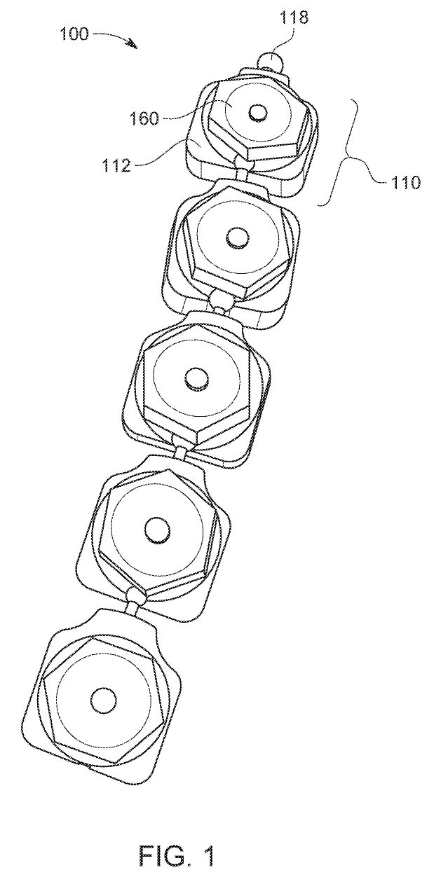

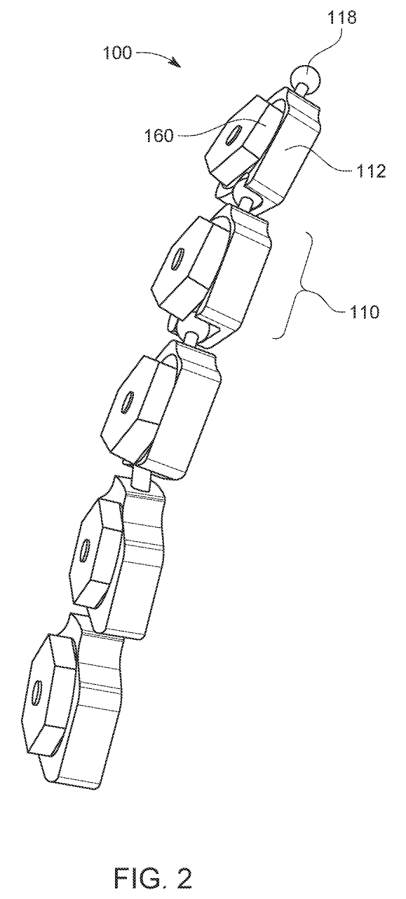

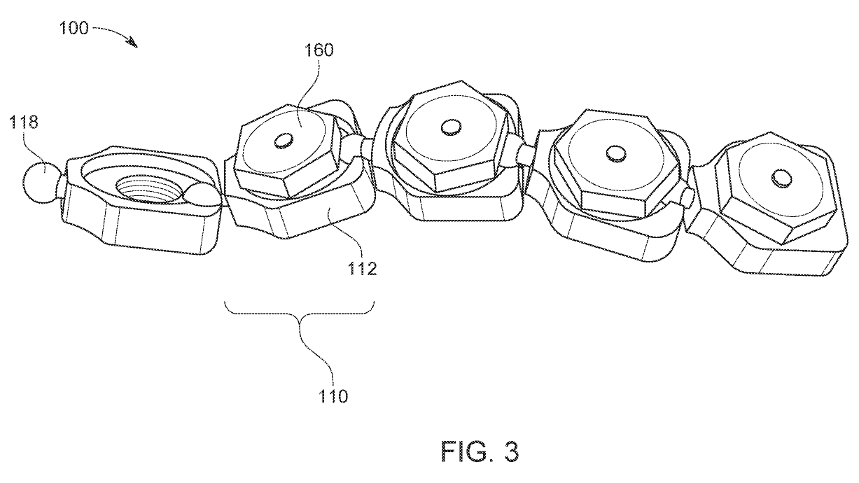

Referring to FIGS. 1, 2, and 3, multiple isometric views of the self- contouring plate with fastener compression are shown.

The self-contouring plate with fastener compression 100 is shown formed from multiple links 110, each link 110 including a plate 112 and fastener 160. Each link no ends with a head or ball 118.

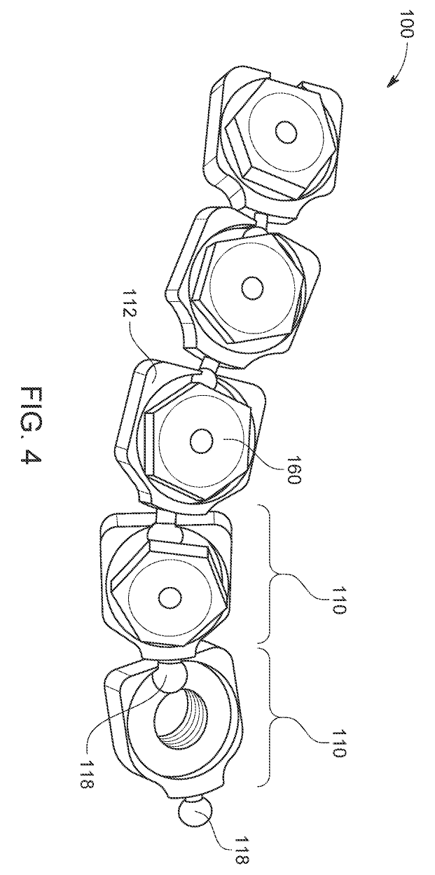

Referring to FIG. 4, a fourth isometric view of the self-contouring plate with fastener compression is shown.

Again shown are links 110 of the self-contouring plate with fastener compression 100. The rightmost link no is shown without its fastener 160.

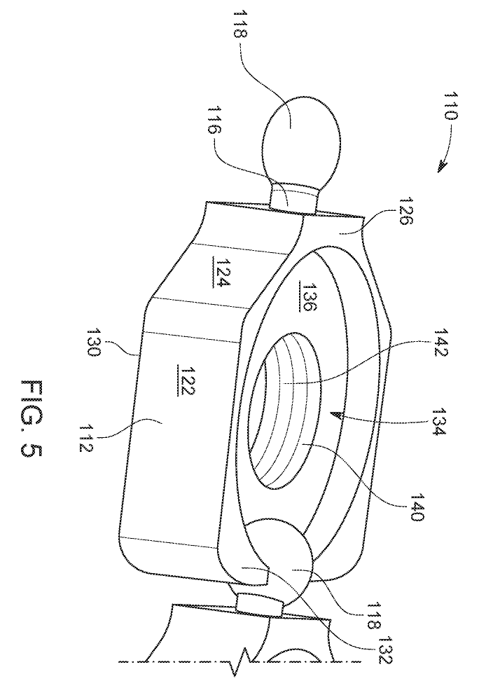

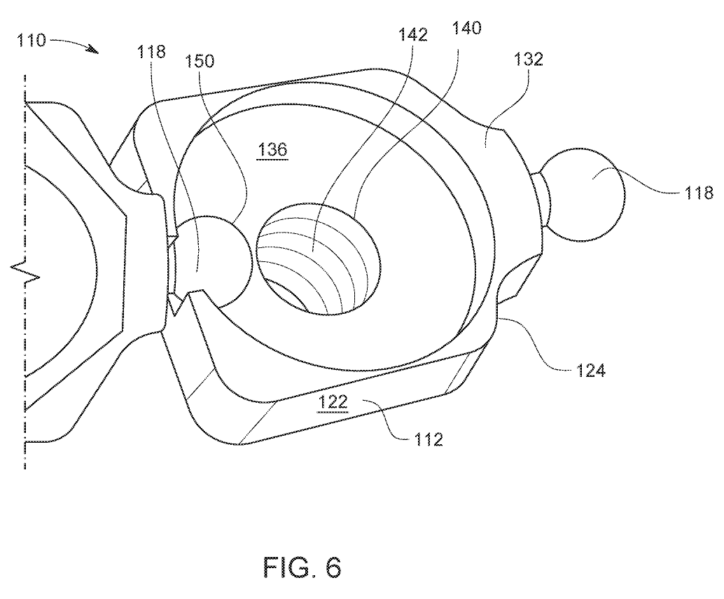

Referring to FIGS. 5 and 6, first and second detail views of the self- contouring plate with fastener compression are shown.

A link no is shown. Each link no is formed from a plate 112. The plate includes outer walls 122, including tapering walls 124 that lead to a neck n16 and head n18. The plate 112 includes an upper surface 132 and a lower surface 130. There is a fastener recess 134 into the upper surface 132, creating a lower, recessed surface 136 into which the fastener 160 fits (see FIG. 1). Each head or ball 118 of a link 112 fits into the socket 150 of the next link 110. The socket 150 is preferably one-half spherical shape that corresponds to the radius of the spherical head 118. Compression of the head 118into the socket 150 will fix the position of a first plate 112 with respect to its adjacent plate 112.

Also shown is the hole 140 through the plate 112, with interior threading 142 that interfaces with the fastener i6o (see FIG. 1).

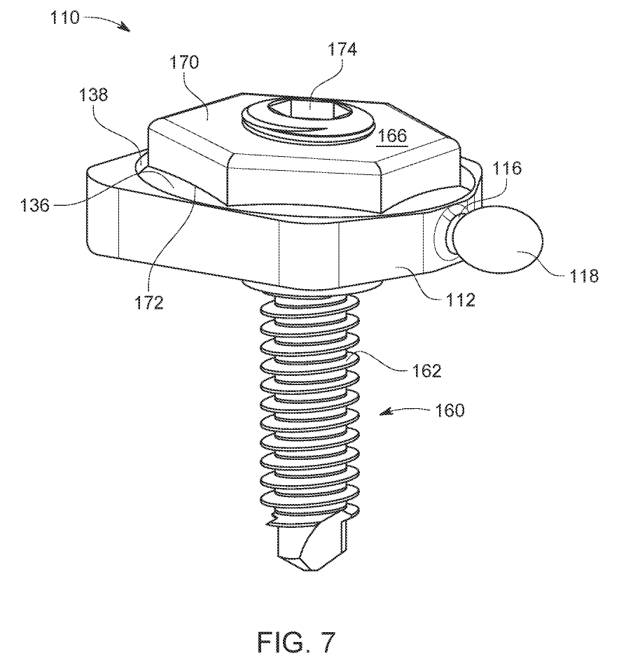

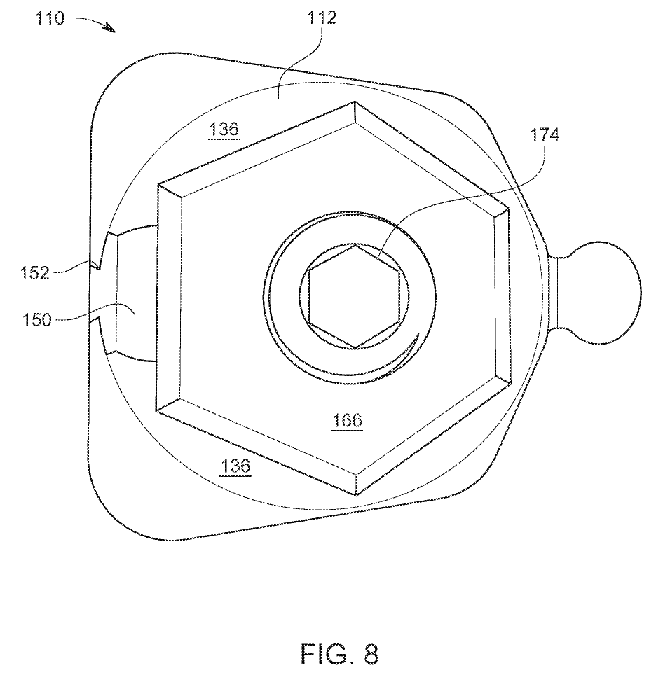

Referring to FIGS. 7 and 8, third and fourth detail views of the self- contouring plate with fastener compression are shown.

Each link 110 is shown with the fastener 160. Each fastener 160 includes a lower set of bone threads 162 that will interface with the bone of the patient. Each fastener 160 further includes an upper set of machine threads 164 that interface with the interior threading 142 of hole 140 (see FIG. 6) in the plate 112.

In a first embodiment, the compression nut 166 is integrated into the faster 160. As the fastener 160 is threaded into the bone, initially the lower bone threads 162 will bite into a hole in the bone, and then as the fastener 160 moves through the plate 112, the upper set of machine threads 164 thread into the plate 112 as the bone threads 162 continue to screw into the bone.

In a second embodiment, the machine threads 164 further interface with the compression nut 166. In this embodiment, the fastener 160 optionally includes a tool interface 174, here shown as a female hex depression, to allow rotation of the fastener 160 separately from the compression nut 166.

In this second embodiment, the compression nut 166 can be tightened with respect to the rotational position of the fastener 160.

The lower surface 172 of the compression nut or head 166 compresses the ball 118 (see FIG. 1) against the inside of the socket 150. This compression fixes the position of each plate 112 with respects to its neighboring plate. The amount of compression available can be set by the position of the recessed surface 136 with respect to the depth of the socket 150. A deeper socket 150 or a higher recessed surface 136 will reduce compression by preventing downward travel of the head or nut 166, correspondingly, a shallower socket 150, or a deeper recessed surface 136 will allow additional compression of the compression nut against the ball 118. The socket 150 optionally includes a neck slot 152 that corresponds to the neck 116 adjacent to the head 118. This tapering area, or neck slot 152, further contains the ball, and can act to limit rotation of the ball 118, controlling the position of the plate 112 with respect to the adjacent plate 112.

Equivalent elements can be substituted for the ones set forth above such that they perform in substantially the same manner in substantially the same way for achieving substantially the same result.

What is claimed is:

1. A device that is adjustable to fit a shape of a surface of a bone to close a fracture, the device comprising:
a series of plates that connect to each other via ball-and-socket joints;
each ball-and-socket including a ball and a socket; and
a single fastener for each plate of the series of plates;
wherein the single fastener compresses the ball-and-socket joint of a given plate of the series of plates, and compresses the given plate against the surface of the bone.

2. The device of claim 1, wherein:
the single fastener further comprises a head; and
a lower surface of the head of the single fastener compresses the ball into the socket to fix a position of a plate with respect to a neighboring plate.

3. The device of claim 2, wherein:
each plate of the series of plates further comprises an upper surface and a recess with a recessed surface;
the lower surface of the head resting against the recessed surface when in its seated position.

4. The device of claim 1, wherein each ball-and-socket joint allows rotation in three degrees of freedom until locked by compression.

5. The device of claim 1, wherein the socket further comprises a neck slot configured to receive a neck portion connecting the ball to a plate of the series of plates.

6. The device of claim 1, wherein the socket comprises a partial spherical cavity corresponding to a radius of the ball.

7. The device of claim 1, wherein the single fastener includes a first set of threads for interfacing with the bone and a second set of threads for interfacing with a plate of the series of plates.

8. A device for stabilizing a fracture in a bone comprising:
a series of plates connected by ball-and-socket joints, each ball-and-socket joint including a ball and a socket, each plate of the series of plates having:
a first setting where their position is adjustable with respect to one another;
a second setting where their position is fixed; and
a fastener;
wherein to change from the first setting to the second setting,
the fastener compresses the ball into the socket while simultaneously affixing the plate to a surface of a bone.

9. The device for stabilizing a fracture in a bone of claim 8, wherein:the fastener further comprises a head; anda lower surface of the head of the fastener compresses the ball into the socket to fix a position of a plate with respect to a neighboring plate.

10. The device for stabilizing a fracture in a bone of claim 9, wherein:each plate of the series of plates further comprises an upper surface and a recess with a recessed surface;the lower surface of the head resting against the recessed surface when in its seated position.

11. The device for stabilizing a fracture in a bone of claim 8, wherein each ball-and-socket joint allows rotation in three degrees of freedom until locked by compression.

12. The device for stabilizing a fracture in a bone of claim 8, wherein the socket further comprises a neck slot configured to receive a neck portion connecting the ball to a plate of the series of plates.

13. The device for stabilizing a fracture in a bone of claim 8, wherein the socket comprises a partial spherical cavity corresponding to a radius of the ball.

14. The device for stabilizing a fracture in a bone of claim 8, wherein the fastener includes a first set of threads for interfacing with the bone and a second set of threads for interfacing with a plate of the series of plates.

15. A self-contouring plate system for bone fractures comprising:
a plurality of rigid plate elements, each plate element comprising:
a plate body having an upper surface;
a ball extending from the plate body;
a socket formed in the plate body, the socket having a socket depth;
a fastener hole extending through the plate body; and
a fastener recess formed in the upper surface, the fastener recess having a predetermined depth relative to the socket depth;
a fastener having a fastener head with a lower surface;
wherein the ball of a first plate element is received in the socket of a second plate element;
wherein the predetermined depth of the fastener recess and the predetermined depth of the socket controls a compression distance between the lower surface of the fastener head and the ball when the fastener is fully seated; and
wherein the compression distance determines a compression force applied by the lower surface of the fastener head against the ball to lock the ball within the socket.

16. The self-contouring plate system for bone fractures of wherein:
the fastener further comprises a compression nut; and
a lower surface of the compression nut of the fastener compresses the ball into the socket to fix a position of a plate with respect to a neighboring plate.

17. The self-contouring plate system for bone fractures of claim 16, wherein:
each plate of the plurality of rigid plate elements further comprises an upper surface and a recess with a recessed surface;
the lower surface of the compression nut resting against the recessed surface when in its seated position.

18. The self-contouring plate system for bone fractures of wherein each ball-and-socket joint allows rotation in three degrees of freedom until locked by compression.

19. The self-contouring plate system for bone fractures of claim 15 wherein the socket further comprises a neck slot configured to receive a neck portion connecting the ball to a plate of the plurality of rigid plate elements.

20. The self-contouring plate system for bone fractures of claim 15 wherein the socket comprises a partial spherical cavity corresponding to a radius of the ball.
